(19) United States
(12) Patent Application Publication
(10) Pub. No.: US 2018/0120254 A1
(43) Pub. Date: May 3, 2018

(54) TREATMENT AND DIAGNOSTIC USING miRNA, PROTEIN AND GENE BIOMARKERS USING QUANTUM DOT FIELD-EFFECT TRANSISTOR (FET) SENSOR PLATFORM

(71) Applicant: Anjana Jain, Silver Springs, MA (US)

(72) Inventor: Anjana Jain, Silver Springs, MA (US)

(21) Appl. No.: 15/657,809

(22) Filed: Jul. 24, 2017

Related U.S. Application Data
(60) Provisional application No. 62/365,422, filed on Jul. 22, 2016.

(51) Int. Cl. G01N 27/414; H01L 29/12; H01L 29/10; H01L 29/49; G01N 33/543; G01N 33/574; C12Q 1/6886; C12Q 1/6825

(52) U.S. Cl. CPC G01N 27/4146; H01L 29/127; H01L 29/1025; H01L 29/495; G01N 33/5438; G01N 2800/28; G01N 33/57484; C12Q 1/6886; C12Q 1/6825; C12Q 2600/178; G01N 2800/2821; G01N 27/4145

(57) ABSTRACT

An array of biosensors diagnosing biomarkers device and a drug delivery vehicle system including a plurality of biosensor arrays for diagnosing biomarkers concentrations, and a delivery vehicle dispensing drug, a electronic interface, a plurality of algorithms to relate biomarker concentrations and drug dispensed, wherein biosensors in said plurality of biosensor arrays are constructed from quantum dot field-effect transistors, and wherein one or more layers of cladded quantum dots are assembled in the channel, gate, and channel and gate regions of FETs, and wherein quantum dots are functionalized by DNA aptamers, antisense oligoneuclotides (ASOs), and DNAs, to sense biomarkers concentrations comprising at least one of proteins, miRNAs, and genes, and wherein the concentrations of biomarkers changes and their values change the magnitude of drain current as a function of time, and wherein the drain current signal is processed by an electronic interface.

### CROSS REFERENCE TO RELATED APPLICATIONS

This application is claims priority to and benefit of the filing date of U.S. Provisional Patent Application No. 62/365,422, the contents of which are incorporated herein by reference in its entirety.

### FIELD OF THE INVENTION

The present invention applies to micro RNA (miRNA), protein and gene biomarkers and more and more particularly to quantum dot (QD) gate field-effect transistors (FETs) configured to provide information on levels of micro RNA (miRNA), DNA, protein and gene biomarkers in body fluid and/or tissue.

### BACKGROUND OF THE INVENTION

This invention describes the use of quantum dot (QD) gate field-effect transistors (FETs) configured as biomarker sensor arrays, providing information on levels of proteins, genes, and micro RNAs (miRNAs) and DNAs in body fluids and tissues. Once biomarker concentrations are measured and their variations (up or down regulation) over time is determined, a treatment protocol to administer a miRNA, combination of multiple miRNAs, proteins and their combinations is made. In the case of up regulation of a miRNA a dose of antisense oligonucleotides (ASO) inhibitor is given. In case a protein, for example P53 is down regulated, a combination of miRNA34a and ASO21 is given. The concentration levels of various species including a wide range of proteins, DNAs, miRNAs, genes changes as a function of time before a disease like cancer is manifested. An array of quantum dot FET sensors on a chip provide the information via an electronic or optical interface to an external monitoring unit. The unit in turn is interfaced to a drug delivery nanocarrier vehicle. The nanocarrier vehicle is selected one of functionalized nanoparticles, nanofibers. These may be complexed or encapsulated.

Proteins, miRNAs, and genes, which are bound to their recognition element that is functionalized to the quantum dots, have a direct effect on the FET drain current levels as different concentrations of these species are immobilized on functionalized QDs. In one embodiment, miRNA sensing is carried out by its binding to an existing complementary miRNA strand functionalized onto the quantum dots located in the gate region. This is in contrast to conventional FET-based DNA detection done by DNA functionalization to gold gated p-channel FETs via gold-thiol.

### SUMMARY OF THE INVENTION

This invention combines diagnostic screening and treatment of cancer at various stages of manifestation. Electronic sensors using quantum dot (QD) gate and/or channel field-effect transistors (FETs) configured as biomarker sensor arrays, provide information on levels of proteins, genes, and micro RNAs (miRNAs) and DNAs in body fluids and tissues. Protein, Genes, RNA and miRNA sensing is done by their binding to antibodies or DNA aptamers, which are functionalized to SiOx-cladded Si quantum dots (located in the gate region or channel region of FETs) prior to sensing. Once biomarker concentrations are measured and their variations (up or down regulation) over time is determined, a treatment protocol is made. In turn a dose of miRNA, combination of multiple miRNAs, proteins and their combinations is made to mitigate RNA and protein levels above normal values. In the case of up regulation of a miRNA a dose of antisense oligonucleotides (ASO) inhibitor is given. In case a protein, for example P53 is down regulated, a combination of miRNA34a and ASO21 is given. The concentration levels of various species including a wide range of proteins, DNAs, miRNAs, genes changes as a function of time before a disease like cancer is manifested. The sensed averaged electronic signals from a sub-sensor array dedicated to a particular miRNa or protein or gene is sent to an external unit via an electronic interface located on the chip platform hosting the biomarker sensors. RF or optical transmission can be used as described in a US patent application (Jain et al, Ser. No. 11/862,866) [11].

Silane functionalized quantum dots serve as detection site for the miRNA, protein and genes. In one embodiment, a number of microRNAs (miRNA), ASOs, as well as proteins (such as NOTCH 1, SIRT 1, P53) sensing is done with the functionalization of a known reference nucleotide sequence on to quantum dots which are located in the gate region or channel region of the FET. The target complementary oligonucleotide strand (e.g. miRNA21) attaches to the reference oligonucleotide and produces a change in the drain current of the FET depending on the concentration. The ASO concentrations can be detected using FET having miRNA21. In an embodiment, oligo-based miRNA aptamers/strands specific to a certain protein (such as NOTCH1, CXCL-10) are functionalized on QD surface. The concentration of ASO and proteins are is detected by the change in current in the FET channel. An array of sensors are used to get an average value. The sensed signal from various devices, disclosed here, can be retrieved by signal processing and Detection of miRNAs, genes and protein biomarkers are significant in the diagnosis of cancer as well as traumatic brain injury and other neural disorders.

A method of diagnosing biomarkers and delivering a drug, wherein the biomarkers include proteins, miRNAs, antisense oligoneuclotides (ASOs), DNA/genes, the method including diagnosing biomarkers, wherein diagnosing is performed by detecting biomarker concentrations as a function of time in at least one of a body fluid and tissue, and wherein biomarker concentrations are determined by a plurality of biosensors, and wherein at least one of the plurality of biosensors include quantum dot based field-effect transistor sensing elements having quantum dots, wherein the quantum dots are functionalized to sense concentrations of at least one of proteins, miRNAs, ASOs, DNAs and genes, and wherein the biomarker concentration changes the drain current in a proportionate manner, and wherein the changed current proportional to biomarker concentration information is signal processed outside the body using body fluids, or using implanted biosensors where the signal is transmitted via wires transcutaneously or wireless via a RF or optical transmitter to an external unit to display the biomarker levels, and wherein quantum dots are disposed in one of transport channel region, gate region, gate and transport channel regions, and wherein quantum dots are functionalized with recognition elements comprising protein aptamers, ASO strands, RNA and DNA strands, and wherein respective biomarkers bind, and wherein drug comprises at least one or more of proteins, anti-sense oligoneuclotides (ASOs), genes and DNAs, and wherein dosage of drugs are based on concentration of proteins and ASOs which up and down regulate concentration of proteins, miRNAs and DNA levels in body fluids and tissues at designated sites, and wherein a nanocarrier vehicle is used to load the drug, and wherein the nanocarrier vehicle is one selected from Si nanofibers, SiOx-coated Si nanowires, polymer nanofibers, re-absorbable nanofibers, and wherein nanofibers are functionalized to deliver ASOs, proteins, miRNAs and their combinations.

An array of biosensors diagnosing biomarkers device and a drug delivery vehicle system including a plurality of biosensor arrays for diagnosing biomarkers concentrations, and a delivery vehicle dispensing drug, a electronic interface, a plurality of algorithms to relate biomarker concentrations and drug dispensed, wherein biosensors in said plurality of biosensor arrays are constructed from quantum dot field-effect transistors, and wherein one or more layers of cladded quantum dots are assembled in the channel, gate, and channel and gate regions of FETs, and wherein quantum dots are functionalized by DNA aptamers, antisense oligoneuclotides (ASOs), and DNAs, to sense biomarkers concentrations comprising at least one of proteins, miRNAs, and genes, and wherein the concentrations of biomarkers changes and their values change the magnitude of drain current as a function of time, and wherein the drain current signal is processed by an electronic interface, and wherein first algorithm determines the concentrations of various biomarkers, and wherein delivery vehicle comprises one or more of nanoparticles, SiOx-Si quantum dots, polymer quantum dots and metallic quantum dots, and wherein nanoparticles and quantum dots are assembled on nanofibers, and wherein nanofibers are selected from polymer, silicon nanowires, quartz, metal and ceramic, and wherein assembled SiOx-Si quantum dots on nanofibers and functionalized with drug comprising of one or more selected from proteins ASOs, DNAs and genes, and wherein the combination of ASOs, miRNAs, proteins, and genes is based on second algorithm, and wherein the said combination is drug that administered at a site, and wherein concentration of biomarkers after a time interval is measured by another set of array of biosensors diagnosing biomarkers device using freshly functionalized quantum dot array FETs, and where in a new cycle of measurements of biomarker concentrations and drug delivery vehicle follows

### BRIEF DESCRIPTION OF THE FIGURES

The foregoing and other features and advantages of the present invention should be more fully understood from the accompanying detailed description of illustrative embodiments taken in conjunction with the following Figures in which like elements are numbered alike in the several Figures:

FIG. 1 shows an Au gate p-MOSFET used for DNA, RNA, or MicroRNA sensing, in accordance with the prior art.

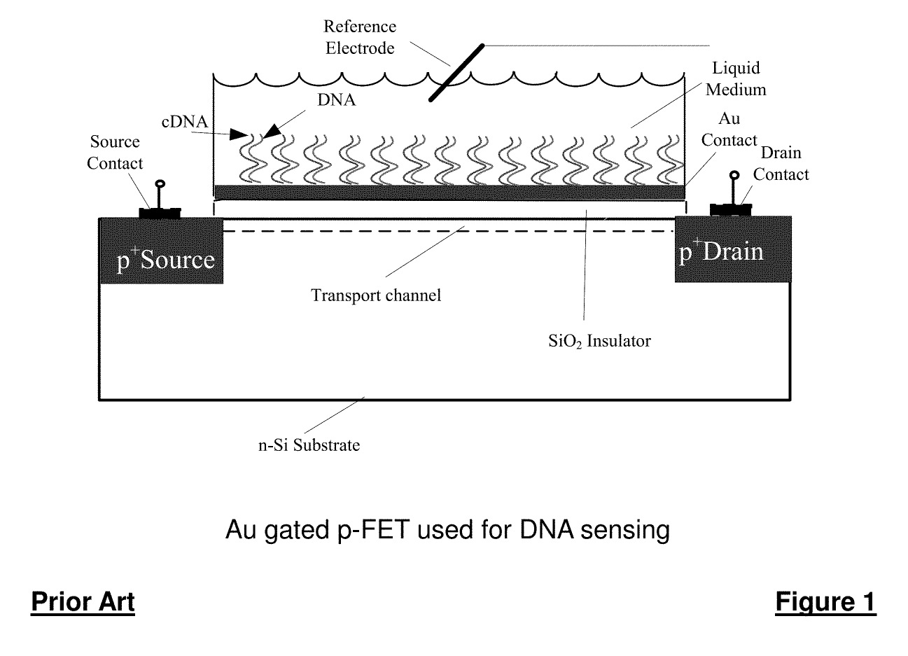

FIG. 2 shows a cross-sectional schematic of an ion-sensitive FET configured for DNA hybridization sensing or gene sequencing, in accordance with the prior art.

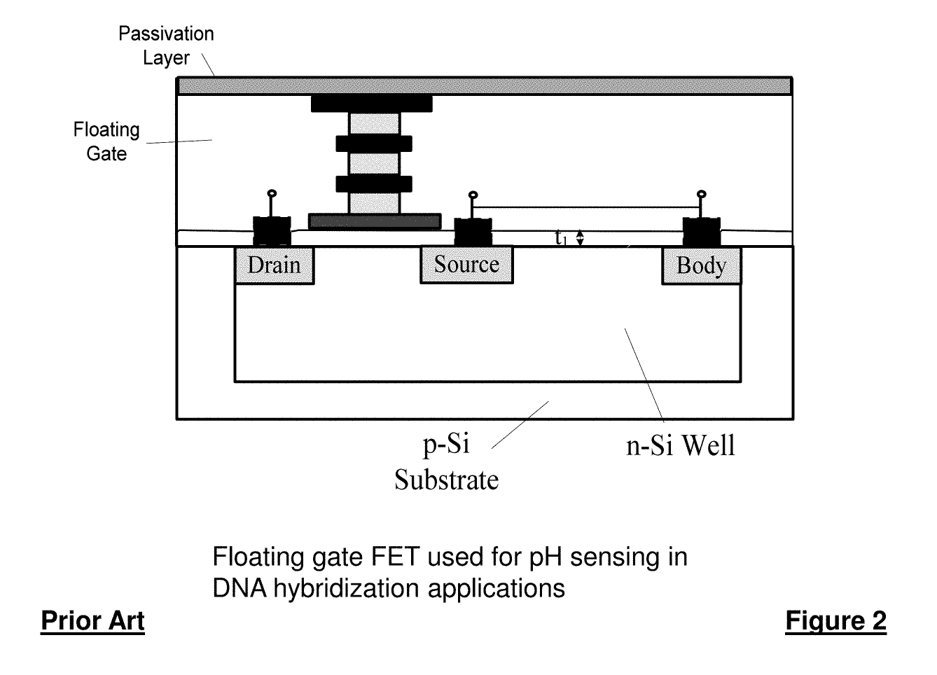

FIG. 3 shows a quantum dot gate FET for DNA, RNA, MicroRNA sensing and sequencing applications, and (inset) silane components for chemical modification of QDs, in accordance with the prior art.

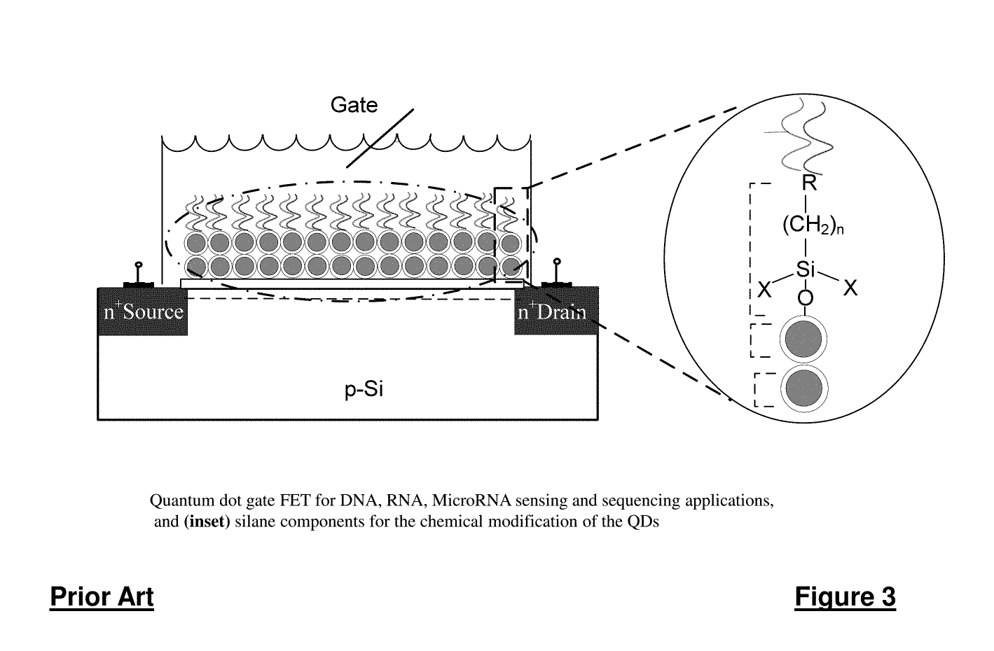

FIG. 4 shows a QD-FET sensor array to detect biomarkers and develop an algorithm/protocol to up and down regulate miRNAs and proteins, in accordance with one embodiment of the present invention.

FIG. 5 shows a block diagram showing an overall diagnostic screening and nanocarrier delivery, in accordance with one embodiment of the present invention.

FIG. 6 is a schematic block diagram for overall cancer screening based on miRNA(i) concentration C(i), protein concentration C(j), and gene concentration C(k), where, variable i, j, and k refer to various species, in accordance with one embodiment of the present invention.

FIG. 7a shows sensor sub-arrays on a chip platform, in accordance with one embodiment of the present invention.

FIG. 7b shows a quantum dot channel FET sensor element for detecting miRNAs, proteins, ASO, genes, in accordance with one embodiment of the present invention.

FIG. 7c shows a quantum dot gate FET biosensor, in accordance with one embodiment of the present invention.

### DETAILED DESCRIPTION OF THE INVENTION

This invention combines diagnostic screening and treatment of cancer at various stages of manifestation. Electronic sensors using quantum dot (QD) gate and/or quantum dot channel field-effect transistors (FETs), configured as biomarker sensor arrays, providing information on levels of proteins, genes, and micro RNAs (miRNAs) and DNAs in body fluids and tissues. Protein, Genes, RNA and miRNA sensing is done by their binding to antibodies or DNA aptamers and antisense oligonucleotides (ASOs), which are functionalized to SiOx-cladded Si quantum dots (located in the gate region or channel region of FETs) prior to sensing. Once biomarker concentrations are measured and their variations (up or down regulation) over time is determined, a treatment protocol is made. In turn a dose of miRNA, combination of multiple miRNAs, proteins and their combinations is made to mitigate RNA and protein levels above normal values. In the case of up regulation of a miRNA a dose of antisense oligonucleotides (ASO) inhibitor is given. In case a protein, for example P53 is down regulated, a combination of miRNA34a and ASO21 is given. The concentration levels of various species (including a wide range of proteins, DNAs, miRNAs, genes) changes as a function of time before a disease like cancer is manifested. This sensing method can be used before and after manifestation of cancer in a tissue. This technique is also applicable to other diseases such as Alzheimer and traumatic brain injury.

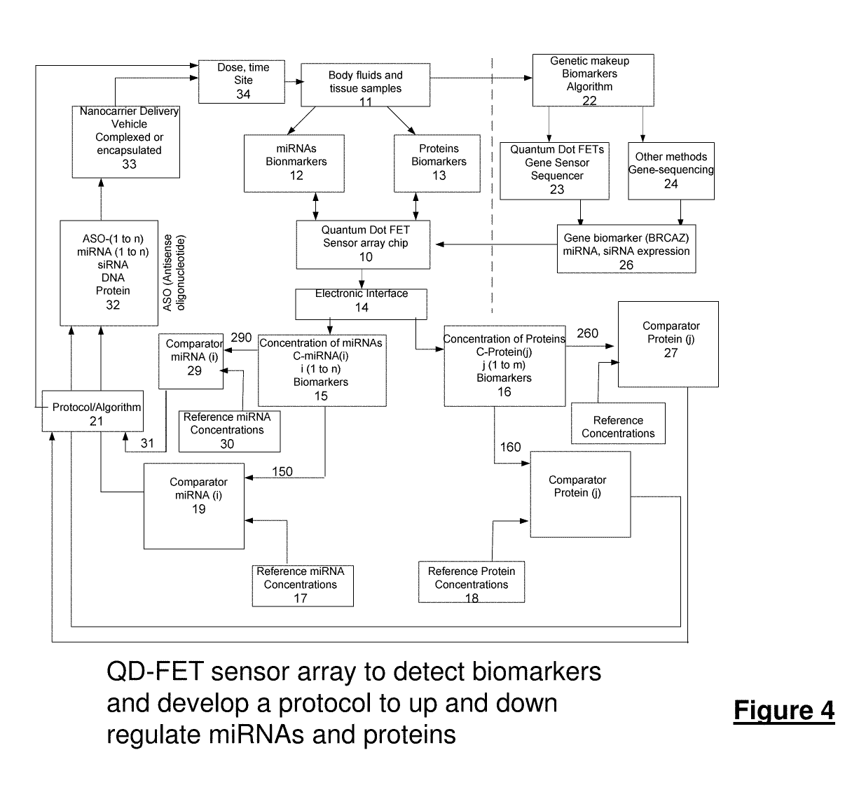

Referring to FIG. 4, a QD-FET sensor array to detect biomarkers and develop a protocol to up and down regulate miRNAs and proteins in accordance with one embodiment of the invention is shown. Here, quantum dot sensor array 10, embedded in the gate or channel of a field effect transistor (FET), senses miRNAs 12 which bind to antisense oligonucleotides (ASOs) functionalized to the SiOx-cladded-Si quantum dots. Similarly, designated proteins 13 bind to aptamer strands functionalized to quantum dots in certain areas of the sensor array. The drain current of the QD-FET sensor varies as a function of miRNA and protein concentrations for a given gate voltage VG and drain voltage VD. The electrical signal represented by current and voltage characteristics is amplified, if necessary, and is processed in an electronic interface unit 14. The miRNA concentration data is stored in ‘as is’ form as well as in processed form (e.g. averaged over a period of time, or rate of change in concentration as a function of time) in block 15. Similarly, the protein data is processed in block 16. The miRNA concentration information 150 is compared using a look up table (LUT) in block 19 with respect to a reference level 17 which depends on personal health care history and the information is fed to the protocol algorithm block 21. Similarly, the designated protein concentrations 160 are compared in block 20 using a look up table (LUT) with respect to a reference level 18 which depends on personal health care history and the information is fed to the protocol algorithm block 21.

In a similar manner, body fluid sample 11 is interrogated for genetic makeup using an algorithm represented in block 22. Gene sequencing is achieved either by quantum dot FET gene sequencer method/chip 23 or conventional techniques represented by block 24. Gene biomarker(s) (BRCAZ) responsible for designated miRNAs and siRNAs and protein expressions (block 26) are separated. The protein and RNA information for this block 26 is obtained using sensor array of block 10. The protein concentrations corresponding to gene biomarkers 260 are compared in comparator block 27 using a reference level 28 information. Designated protein concentrations 260 are compared in block 27 using a look up table (LUT) with respect to reference levels 28 which depends on personal health care history and the information is fed to the protocol algorithm block 21. Similarly, miRNAs and siRNAs levels 290 corresponding to genetic makeup/sequencing and measured by QD-FET sensors in block 10 are compared in block 29 comparator electronics against a reference level 30. The output 31 is fed to the protocol/algorithm block 21.

Once biomarkers [e.g. miRNAs, genes (BRCAZ), and proteins] concentrations and their time variation trends are determined for screening of a particular cancer or disease using the quantum dot sensor array 10 along with its interface unit 14, an algorithm 21 (first algorithm) and protocol determines the dose levels 32 of various ASOs and proteins depending on the delivery vehicle 33. Finally, a dose is decided and administered. This is represented by block 34. The concentration of various miRNAs (e.g. miRNA34a, miRNA 21), proteins (e.g. NOTCH 1, SIRT 1, P53), gene (e.g. BRACZ) is compared with reference values in Look Up Tables (LUTs).

In one embodiment, the protocol/algorithm includes controlling the down regulation of miRNA34a by administering at appropriate site the miRNA34a. In case miRNA21 is up-regulated, ASO21 is provided to inhibit it. The delivery vehicle for ASO, miRNAs, proteins in the form of nanocarriers-complexed or encapsulated is shown in block 33. Nanocarriers include nanofibers of appropriate material with functionalized SiOx-cladded Si quantum dots in one embodiment. The dose (combining ASOs, miRNAs and proteins) are administered following an algorithm 34 (second algorithm). Here, the history of doses and site is recorded in a look-up-table. The loop is closed by taking body fluid samples at a later time as represented by block 11. In another embodiment, a nanofiber based drug delivery vehicle may also serve as a rail to retract tumor cells such, as in glioblastoma.

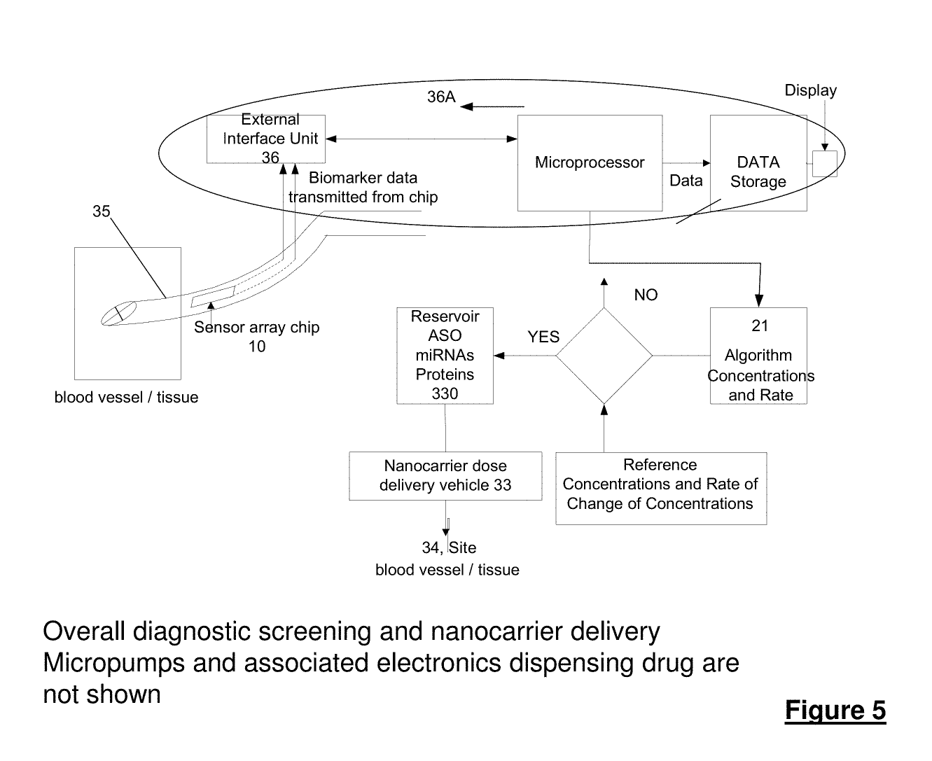

Referring to FIG. 5, a block diagram describing an overall diagnostic screening and nanocarrier based delivery system is shown in accordance with one embodiment of the invention. The sensor array data [concentrations C(i), C(j), and C(k)], obtained from chip 10 (FIG. 4) implanted in blood vessel or externally using serum/plasma, is transmitted to an external unit 36. The microprocessor 140 in the external unit processes and stores the data in a dedicated storage 141 (and if needed could be displayed in a display 142). The algorithm 21 finds the trends of concentration changes over a period, if needed at predetermined intervals. The algorithm 21 compares the reference levels (initially from healthy person and subsequently from previous data from person under screening) and is used to determine the dose and delivery method.

In one embodiment, a catheter 35 is used which also houses the sensor array chip 10 and its associated electronics 14 and RF or optical transmitter. The transmitter communicates with an external unit 36 in turn interfaced with a microprocessor 140, data storage 141 and display 142. The processed data of concentrations and trends of various biomarkers and reference values are assessed by the algorithm 21 stored either in the external unit processor or a separate computing/microcontroller device. The combination dose 330 (e.g. ASOs and proteins) is functionalized on nanocarriers (or without functionalization) is delivered to the site 34.

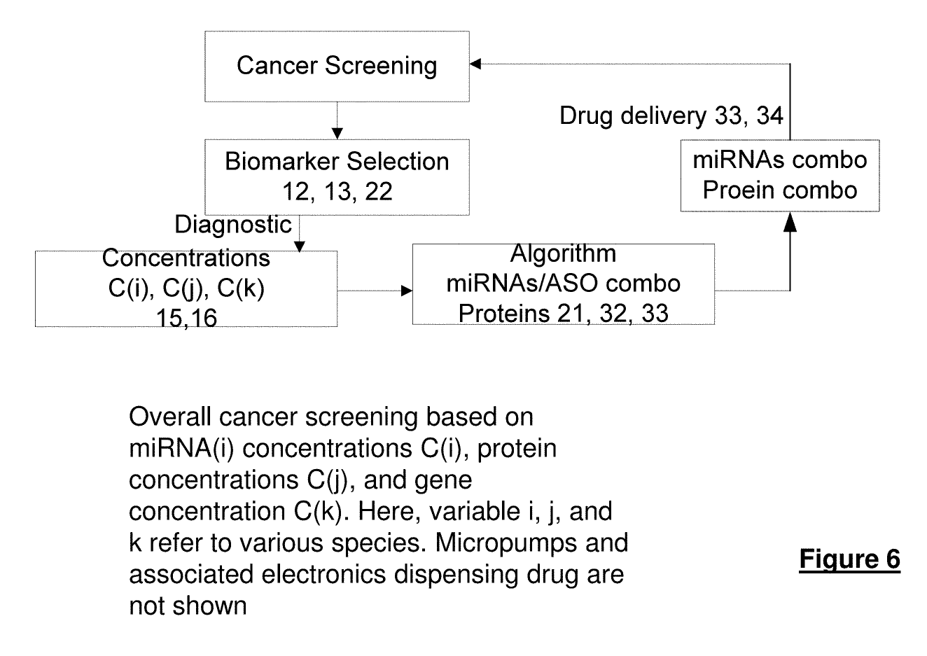

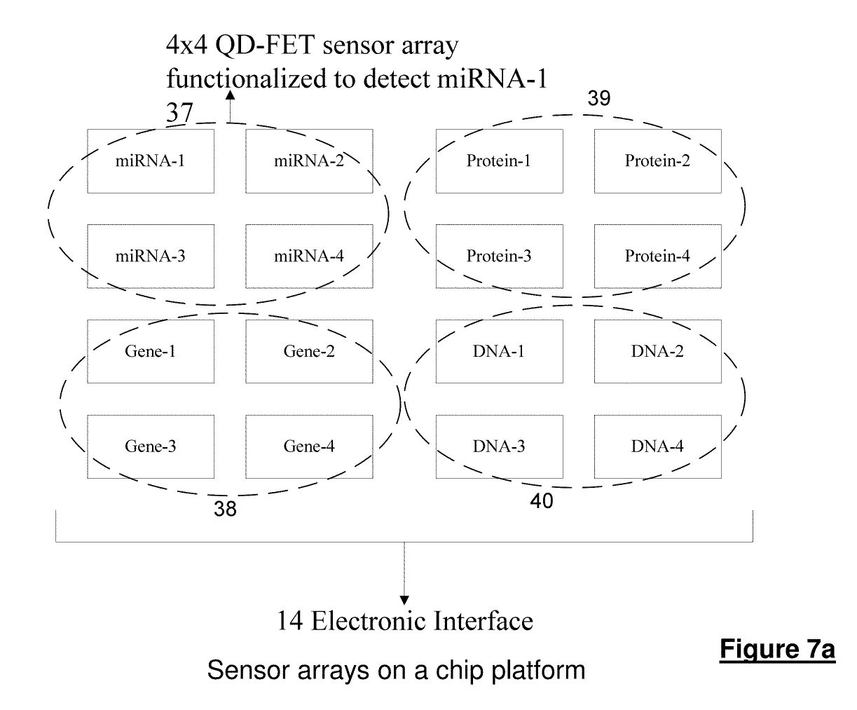

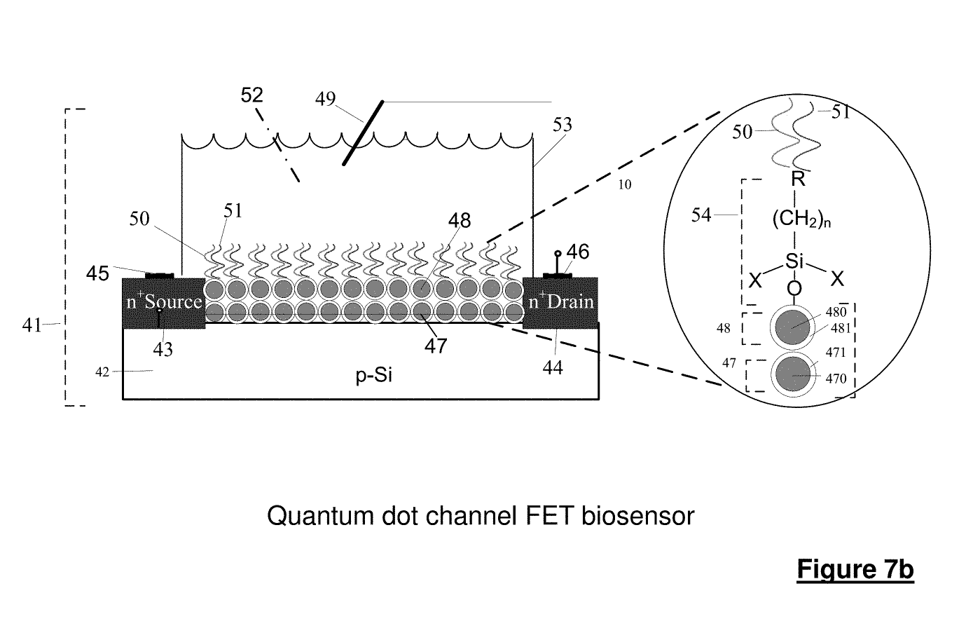

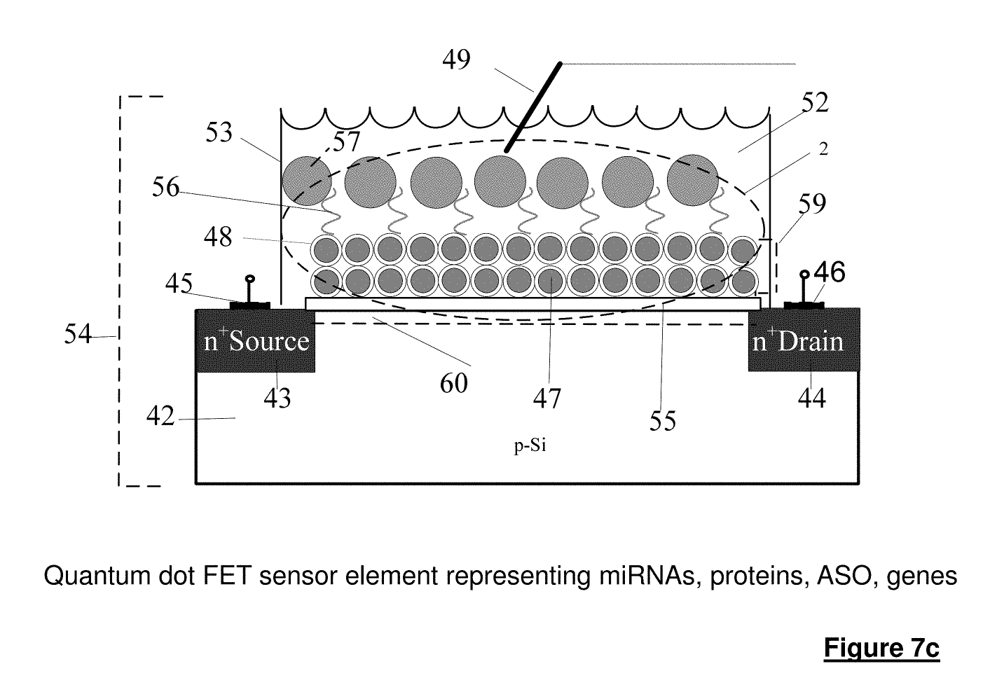

Referring to FIG. 6, a schematic block diagram for overall cancer screening and treatment is shown and is based on finding biomarkers 12, 13, 22 concentrations (15, 16) miRNA(i) C(i), proteins C(j), and gene C(k). Here, variable i, j, and k refer to various species. This is part of diagnostics using quantum dot based sensor arrays (shown in FIG. 7). An algorithm (21, 32, and 33) is used to find up and down regulation of miRNAs using ASOs. This leads to drug delivery vehicle and administration of drug at the chosen site(s).

Referring to FIG. 7a, sensor arrays on a chip platform 10 are shown, where in one embodiment each miRNA concentration is determined by taking an average over 4×4 or higher sub-sensor array shown here 37. Similarly, gene sequencer 38, protein array 39 and DNA strand array 40 are schematically shown. The subarrays are interfaced with electronic interface 14 and microprocessor 140. These are shown as part of unit 36. In another embodiment, arrays of one type of quantum dot sensors are enclosed in one enclosure having its gate electrode and buffer solution and biomarker solution is added in certain concentration. Similarly, other arrays of quantum dot sensors are enclosed in another enclosure (for example realized in SU8 or PDMS) and having their own gate and designated biomarker is added for concentration level detection.

Referring to FIG. 7b, sensor element 41 is based on quantum dot channel FET for detecting miRNAs, proteins, ASOs, DNAs and genes. Figure shows miRNA strands 51 bind to an ASO strand 50. The ASOs are functionalized to the top of cladded quantum dot layer 48, which along with lower quantum dot layer 47 forms the quantum dot channel of the FET sensor 41. The n-channel FET is realized on a p-Si layer 42. It is shown with its source 43 and drain 44. The source and drain contacts are 45 and 46, respectively. Unlike a conventional FET, here the transport channel which carries the electron current is composed of one or more layers of cladded quantum dots. The gate electrode is 49. The top layer 48 of cladded Si quantum dot has a core 480 and a SiOx cladding 481. The bottom layer of dots 47 has a core 470 and a cladding 471. In this case the oxide cladding 481 is thicker and it also serves as the tunnel oxide (or gate insulator). Direct functionalization process of SiOx-Si QDs has been reported elsewhere, where a single-stranded DNA (ssDNA) thrombin aptamer (5′-GGTTGGTGTGGTTGG-3′-NH₂) is covalently attached to the QD surface, which specifically binds to the protein thrombin. Similarly, aptamers of biomarkers of proteins such as NOTCH 1, SIRT 1, P53 and others can be used for functionalization of cladded Si dots. Other quantum dots may have different chemistry. The ASOs, miRNAs, ssDNA aptamers, and genes/DNA strands are immersed in a buffer 52 which is located in a chamber 53. Source electrical contact 45 and drain contact 46 is electrically isolated from the Pt (or other gate metal) electrode 49. In one embodiment, the drain current I_{DS} is a function of protein and miRNA concentrations. The current is signal processed and the information is transmitted using a RF or optical transducer.

Referring to FIG. 7c, the protein (e.g. NOTCH 1) functionalized to its aptamer in quantum dot gate FET sensor is shown. For application to detect traumatic brain injury level of CXCL-10 and protein tau may be detected. Here, the sensor element 54 is based on quantum dot gate FET for detecting miRNAs, proteins, ASOs, DNAs and genes. Figure shows protein 57 binding to an aptamer strand 56. The aptamers are functionalized to the top of cladded quantum dot layer 48, which along with lower quantum dot layer 47 forms the quantum dot gate of the FET sensor 54. The n-channel FET is realized on a p-Si layer 42. It is shown with its source 43 and drain 44. The source and drain contacts are 45 and 46, respectively. The transport channel 60 forms on top of Si layer 42 and it carries the electron current which is dependent on the gate charge which is controlled by the protein charge. The quantum dot layers 47 and 48 are disposed on a tunnel gate oxide 55. The gate electrode is 49. The top layer 48 of cladded Si quantum dot has a core 480 and a SiOx cladding 481. The bottom layer of dots 47 has a core 470 and a cladding 471. The ssDNA aptamers 56 are immersed in a buffer 52 which is located in a chamber 53. Source electrical contact 45 and drain contact 46 is electrically isolated from the Pt (or other gate metal) electrode 49. The drain current I_{DS} is a function of protein concentrations. The current is signal processed and the information is transmitted using a RF or optical transducer. This can be adapted for genes/DNA strands, miRNAs and other proteins.

Moreover, while the invention has been described with reference to an exemplary embodiment, it will be understood by those skilled in the art that various changes, omissions and/or additions may be made and equivalents may be substituted for elements thereof without departing from the spirit and scope of the invention. In addition, the elements and characteristics of the disclosed embodiments may be combined in whole or in part and/or many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

I claim:

1. A method of diagnosing biomarkers and delivering a drug, wherein the biomarkers are comprising of proteins, miRNAs, antisense oligoneuclotides (ASOs), DNA/genes, the method comprising:
diagnosing biomarkers, wherein diagnosing is performed by detecting biomarker concentrations as a function of time in at least one of a body fluid and tissue, and
wherein biomarker concentrations are determined by a plurality of biosensors, and wherein at least one of the plurality of biosensors include quantum dot based field-effect transistor sensing elements having quantum dots, wherein the quantum dots are functionalized to sense concentrations of at least one of proteins, miRNAs, ASOs, DNAs and genes, and
wherein the biomarker concentration changes the drain current in a proportionate manner, and
wherein the changed current proportional to biomarker concentration information is signal processed outside the body using body fluids, or using implanted biosensors where the signal is transmitted via wires transcutaneously or wireless via a RF or optical transmitter to an external unit to display the biomarker levels, and
wherein quantum dots are disposed in one of transport channel region, gate region, gate and transport channel regions, and
wherein quantum dots are functionalized with recognition elements comprising protein aptamers, ASO strands, RNA and DNA strands, and wherein respective biomarkers bind, and
wherein drug comprises at least one or more of proteins, anti-sense oligoneuclotides (ASOs), genes and DNAs, and
wherein dosage of drugs are based on concentration of proteins and ASOs which up and down regulate concentration of proteins, miRNAs and DNA levels in body fluids and tissues at designated sites, and
wherein a nanocarrier vehicle is used to load the drug, and
wherein the nanocarrier vehicle is one selected from Si nanofibers, SiOx-coated Si nanowires, polymer nanofibers, re-absorbable nanofibers, and
wherein nanofibers are functionalized to deliver ASOs, proteins, miRNAs and their combinations.

2. The method of claim 1, further comprising aptamers, wherein the aptamers are used to detect protein biomarkers related to Alzheimer and traumatic brain injury.

3. The method of claim 1, where an algorithm is used to develop concentrations of various constituents of a combination of ASOs, miRNAs and protein to up and down regulate levels of proteins and miRNAs.

4. An array of biosensors diagnosing biomarkers device and a drug delivery vehicle system comprising:
a plurality of biosensor arrays for diagnosing biomarkers concentrations, and
a delivery vehicle dispensing drug,
a electronic interface,
a plurality of algorithms to relate biomarker concentrations and drug dispensed,
wherein biosensors in said plurality of biosensor arrays are constructed from quantum dot field-effect transistors, and wherein one or more layers of cladded quantum dots are assembled in the channel, gate, and channel and gate regions of FETs, and
wherein quantum dots are functionalized by DNA aptamers, antisense oligoneuclotides (ASOs), and DNAs, to sense biomarkers concentrations comprising at least one of proteins, miRNAs, and genes, and wherein the concentrations of biomarkers changes and their values change the magnitude of drain current as a function of time, and
wherein the drain current signal is processed by an electronic interface, and wherein first algorithm determines the concentrations of various biomarkers, and
wherein delivery vehicle comprises one or more of nanoparticles, SiOx-Si quantum dots, polymer quantum dots and metallic quantum dots, and
wherein nanoparticles and quantum dots are assembled on nanofibers,
and wherein nanofibers are selected from polymer, silicon nanowires, quartz, metal and ceramic, and
wherein assembled SiOx-Si quantum dots on nanofibers and functionalized with drug comprising of one or more selected from proteins ASOs, DNAs and genes, and
wherein the combination of ASOs, miRNAs, proteins, and genes is based on second algorithm, and wherein the said combination is drug that administered at a site, and
wherein concentration of biomarkers after a time interval is measured by another set of array of biosensors diagnosing biomarkers device using freshly functionalized quantum dot array FETs, and where in a new cycle of measurements of biomarker concentrations and drug delivery vehicle follows.

5. The device of claim 4, further comprising,
arrays of quantum dot FET biosensors for diagnosing cancer biomarkers, and wherein a electronic interface and first algorithm determines the concentrations and their time variations of biomarkers comprising proteins, miRNAs, genes, and
wherein nanocarrier delivery vehicle dispensing drug dose comprising one or more selected from proteins, ASOs, miRNAs, and genes, and wherein the drug dose is determined by a second algorithm and,
wherein site to administer nanocarrier delivery vehicle is determined by the second algorithm, and
wherein biomarker concentrations are determined at a later time after a time interval and compared by said electronic interface with the previous concentrations, and
wherein the nanocarrier delivery vehicle drug dose is adjusted to maintain biomarker levels, and
wherein nanocarrier drug dose is dispensed using micropumps, and
wherein micropumps are controlled by said electronic interface and its associated microprocessor.
